SEQUENCE LISTING (5 sequences)

SEQ ID NO: 1 - DNA, 601 bp, Homo sapiens
tgtaccaagcactctgcatctgcttttaaagtcttcagactactgtattagtcatagcctctcagaaggagccacaggaacggcgggacaatggggattaaaggcctttcctttctcttccaggctgcccccaagtgtaactccagcactgtgaggtttcagggattggcagaggggaccaaggggaccatgaaaatggacatggaggatgcggatatgactctgtggacagaggctgagtttgaagagaagtgtacatacattgtgaacgaccacccctgggattctggtgctgatggcrgtacttcggttcaggcggaggcatccttaccaaggaatctgcttttcaagtatgccaccaacagtgaagaggtaagcctctggtttattgacaagaagattggggacctggtgccaaatctccctacttgcccttgaggccttgtatatctctgaaaacctctgagaatctgtaagtatcagtaaataattgattgctctattcaattcttgcattgctttctctttccctaaaccatttccttctcatttcttccagccttcaactgttcctcactaattagtaaacagttaaatattt

SEQ ID NO: 2 - DNA, 801 bp, Homo sapiens
gctagatcataatcacaagacagtgacagcatcacatcctcaaacccaatgtccaggggtttatgtgtctttttaaaaactaagatgctccttctcatctccttgtcgagaacttgattgtgacccatcctgaagccattcacatgtaagaagaatgagaccttcatgagctgcttagaccaggcaaggtttaactaagtcccatggtagggcacaggggcaaagtcaaaattctgggtaagcaactgactgtgcgtgttaggctgctgttaagccaatgtgtgattcagcctaagaataaaatacttgctatgagatccaaaatgggctatttccataatttttttctagtttaatactttgtcccttggaaaaccccatgaataaaatgtgagcaggcrctaaccctaaatgaaatagatttatactggtaattagagcttgttctacattagcaacagtgttgttttttttttttttttttttttttttttagataacattccacggggccaggcatggtggcttacgcctataatcccagcactttgggaggccgaggtggatcacgaggtcaggagttcaagaccagcctgaccaagatggtgaaaccccgtctctactacaaatacaaaaaatagcagggagtgatggggggcgcctgtaagcccagctactcaggaggctgaggcaggagaatcgcttgaacccaggagactgaggttgcagtgagccgagatcacgccactgcactccagcctgggcgacagagcgagactctgtctcaaagaaaataaaaca

SEQ ID NO: 3 - DNA, 701 bp, Homo sapiens
gaaggtggtattcacttcgctaagctccttggcataggcagtttgaacagggctttatcaaattcgtattcaacaagagtagaagcgaaaattgatgactgtgtattacttgaaatgagtcttaatctttcacatttagttctcagggtatgctgatttcctttaggtaaaccatgaacatcagaaagacttttattaacytatgacagggtccccaccccagtatttttccactccattaaaatggaagttttttttttttttttcttttttgagacagagttttgctcttgttgcccagtctggagtgcaatggcacaatctcggctcaccacaacctccacctcccagattcaagcgattcttctgcctcagcctcccaagtagctgggattacaggtgtgcgccaccacgcccagctaattttgtatttttagtagagatggggtttctccatgttggtcaggctggtctcgaacttccgacctcaggtgatccgcccacctcggcctcccaaagtgctgggattacaggcaagagccactgcatccagcttaggctatcttactccagcctaaacagcaattttctatcataaggtctgtactaatgaaaacagaatcacccaaggctgctgtttgttctgtctgtgctgccattgtccgcattttgctgaggaggaaacggaactgcacttttga

SEQ ID NO: 4 - DNA, 503 bp, Homo sapiens
tgcctttgagcaatgccacattccagtggggagtggttcccccacattcaccgcactagggcgggcgtgttgtgcctcgtgcctgcagccacccttgaagtgttgttgttaaacaacagtcatgaactctggaatgtctctggtcatttatagcgtgactgtgtcactctgtaaatgacactgtcaagcttcaaggcctagaatgaagttggtttgcttttcaatagatcatagtcccgcccaaaacaaagcttccaggcttttacaatgtacttcttactctgaaactcctaagagaatagaaatgctagggagataaaatacaggaatagactacaaatcacactccagtcatcttacaggccctttaaaacacatagaacaatttgaccatactatttgcaaataagcagctttctggaaattagctgggctcttcttaaagcaatttttgtcttctctcacycttgtcttgacttttccttctttggttcatttccttc

SEQ ID NO: 5 - DNA, 501 bp, Homo sapiens
tctgcaacctacagcttattatgtactagctatgaaggtaggtttgtggaggattcacgaaacgtaattcccatagtcttgtgttcttttgacccattccttacaagaaaaaagtgcttacaaaatccttcctactgtccctcttacttttaaatccctttcatcaaataggttgagtcaatacatgaatcctgagatttttaacattttttgttctcaagttgtaggttttccattgtattcatgaaagrtaagtcctgcatttttactgattcattcattcattctatgaacatttattacctgcctattatacttcaaacatgggccaagctgggcctgaaggccaggtccaaatatgaccaggaccttgacctcaaagaaatacttgcttcatgaaggagagagcaatgtacaatctgattaatgttattggtctcttttaaataaattggttctattaatatctgctaagatggatcactgaattagaaatactaa

(19) United States
(12) Patent Application Publication
(10) Pub. No.: US 2013/0012604 A1
(43) Pub. Date: Jan. 10, 2013

(54) METHODS OF USING PRDM1 GENETIC VARIANTS TO PROGNOSE, DIAGNOSE AND TREAT INFLAMMATORY BOWEL DISEASE

(75) Inventors: Jerome I. Rotter, Los Angeles, CA (US); Kent D. Taylor, Ventura, CA (US); Stephan R. Targan, Santa Monica, CA (US)

(73) Assignee: CEDARS-SINAI MEDICAL CENTER, Los Angeles, CA (US)

(21) Appl. No.: 13/635,643

(22) PCT Filed: Mar. 16, 2011
(86) PCT No.: PCT/US2011/028694

§ 371 Date: Sep. 17, 2012

Related U.S. Application Data
(60) Provisional application No. 61/314,331, filed on Mar. 16, 2010.

(51) Int. Cl. C12Q 1/68; G01N 27/447; C40B 30/04; A61K 35/00; A61P 1/04

(52) U.S. Cl. 514/789; 435/6.11; 506/9; 204/461

(57) ABSTRACT

Methods of predicting the development of medically refractory ulcerative colitis (MR-UC) in a patient by determining the presence or absence of one or more risk variants, where the presence of one or more risk variants is indicative of a severe and/or aggressive form of ulcerative colitis are disclosed.

### CROSS-REFERENCE TO RELATED APPLICATIONS

The present application claims the benefit of the filing date of U.S. Provisional Application No. 61/314,331 filed Mar. 16, 2010, the disclosure of which is incorporated herein by reference in its entirety.

### FIELD OF INVENTION

The invention relates generally to the field of inflammatory bowel disease, specifically to ulcerative colitis.

### BACKGROUND

Crohn's disease (CD) and ulcerative colitis (UC), the two common forms of idiopathic inflammatory bowel disease (IBD), are chronic, relapsing inflammatory disorders of the gastrointestinal tract. Each has a peak age of onset in the second to fourth decades of life and prevalence's in European ancestry populations that average approximately 100-150 per 100,000 (D. K. Podolsky, N Engl J Med 347, 417 (2002); E. V. Loftus, Jr., Gastroenterology 126, 1504 (2004)). Although the precise etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (D. K. Podolsky, N Engl J Med 347, 417 (2002)). Genetic factors play an important role in IBD pathogenesis, as evidenced by the increased rates of IBD in Ashkenazi Jews, familial aggregation of IBD, and increased concordance for IBD in monozygotic compared to dizygotic twin pairs (S. Vermeire, P. Rutgeerts, Genes Immun 6, 637 (2005)). Moreover, genetic analyses have linked IBD to specific genetic variants, especially CARD15 variants on chromosome 16q12 and the IBD5 haplotype (spanning the organic cation transporters, SLC22A4 and SLC22A5, and other genes) on chromosome 5q31 (S. Vermeire, P. Rutgeerts, Genes Immun 6, 637 (2005); J.P. Hugot et al., Nature 411, 599 (2001); Y. Ogura et al., Nature 411, 603 (2001); J. D. Rioux et al., Nat Genet 29, 223 (2001); V. D. Peltekova et al., Nat Genet 36, 471 (2004)). CD and UC are thought to be related disorders that share some genetic susceptibility loci but differ at others.

In UC, while inflammation of the colon is a common feature, patients vary in their age of onset, disease extent and natural history, as well as their response to medical therapies and their need for surgery. Medically refractive ulcerative colitis (MR-UC) requiring colectomy remains a significant challenge in the management of IBD. More than 12% of around 1100 UC patients assessed for more than 5 years developed toxic, fulminant or severe colitis, while another recent population-based study demonstrated that 7.5% of approximately 450 Norwegian UC patients followed up for five years required colectomy.

Blimp-1, a transcriptional repressor encoded by the PRDM1 gene, is a crucial regulator of T cell function. In mice, T-cell specific deletion of Blimp-1 results in altered peripheral T cell homeostasis and development of a fatal inflammatory response targeting specifically the colon. Regulation of T cell responses by Blimp-1 could influence intestinal mucosa homeostasis and could potentially have a role in inflammatory bowel disease pathogenesis and progression.

### BRIEF DESCRIPTION OF THE FIGURES

Exemplary embodiments are illustrated in referenced figures. It is intended that the embodiments and FIGURES disclosed herein are to be considered illustrative rather than restrictive.

FIG. 1 depicts a haplotype map implicating variants at the PRDM1 genetic locus associated with ulcerative colitis. Alleles for PRDM1 Block 1 SNPs rs573869 (SEQ. ID. NO.: 3), rs548234 (SEQ. ID. NO. 4) and rs693612 (SEQ. ID. NO.: 5) are provided as corresponding to positions 19, 22 and 23, respectively.

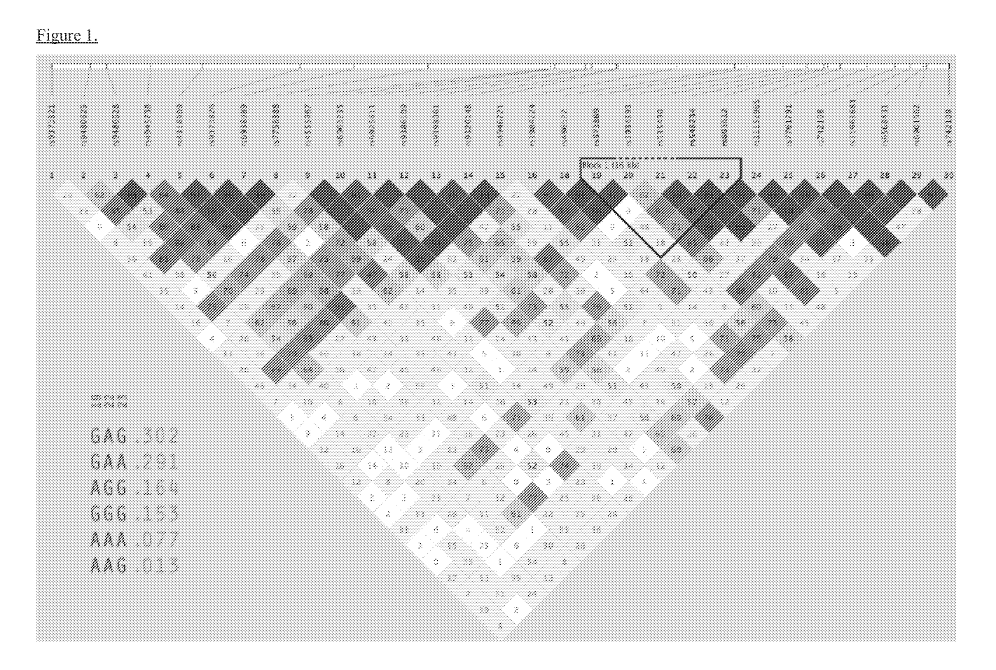

### SUMMARY OF THE INVENTION

In one embodiment, the invention provides a method of prognosing ulcerative colitis (UC) in a subject, comprising: obtaining a sample from the subject, assaying the sample for the presence or absence of one or more risk variants at the PRDM1 genetic locus, and prognosing a severe form of UC based on the presence of one or more risk variants at the PRDM1 genetic locus. The severe form of UC is Medically refractive ulcerative colitis (MR-UC). The risk variants can be selected from the group consisting of SEC. ID. NO.: 1, SEC. ID. NO.: 2, SEQ. ID. NO.: 3, SEQ. ID. NO.: 4, and SEQ. ID. NO.: 5. The presence of one or more risk variants is determined by assaying for the presence of the Blimp-1 protein and/or Blimp1 mRNA. The sample can be whole blood, plasma, serum, saliva, cheek swab, urine, or stool.

In another embodiment, the invention provides a method of diagnosing susceptibility to ulcerative colitis (UC) in a subject, comprising: obtaining a sample from the subject, assaying the sample for the presence or absence of one or more risk variants at the PRDM1 genetic locus, and diagnosing a severe form of UC based on the presence of one or more risk variants at the PRDM1 genetic locus. The severe form of UC is MR-UC. The risk variants can be selected from the group consisting of SEC. ID. NO.: 1, SEC. ID. NO.: 2, SEQ. ID. NO.: 3, SEQ. ID. NO.: 4, and SEQ. ID. NO.: 5. The presence of one or more risk variants is determined by assaying for the presence of the Blimp-1 protein and/or Blimp1 mRNA. The sample can be whole blood, plasma, serum, saliva, cheek swab, urine, or stool.

In a related embodiment, the invention provides a method of diagnosing susceptibility to MR-UC in a subject, comprising: obtaining a sample from the subject, assaying the sample for the presence or absence of one or more MR-UC genetic risk variants, and diagnosing susceptibility to MR-UC based on the presence or absence of one or more MR-UC genetic risk variants. The MR-UC genetic risk variants can be selected from the group consisting of SEC. ID. NO.: 1, SEC. ID. NO.: 2, SEQ. ID. NO.: 3, SEQ. ID. NO.: 4, and SEQ. ID. NO.: 5. The presence of one or more MR-UC genetic risk variants is determined by assaying for the presence of the Blimp-1 protein and/or Blimp1 mRNA. The sample can be whole blood, plasma, serum, saliva, cheek swab, urine, or stool.

In a further embodiment, the invention provides method of treating a subject for Medically refractive ulcerative colitis (MR-UC), comprising: determining the presence or absence of one or more risk variants at the PRDM1 genetic locus, and treating the subject, wherein the one or more risk variants can be selected from the group consisting of SEC. ID. NO.: 1, SEC. ID. NO.: 2, SEQ. ID. NO.: 3, SEQ. ID. NO.: 4, and SEQ. ID. NO.: 5. The presence of one or more risk variants is determined by assaying for the presence of the Blimp-1 protein and/or Blimp1 mRNA. The sample can be whole blood, plasma, serum, saliva, cheek swab, urine, or stool.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

The term “inflammatory bowel disease” or “IBD” refers to gastrointestinal disorders including, but not limited to Crohn's disease (CD), ulcerative colitis (UC), indeterminate colitis (IC), and medically refractive ulcerative colitis (MR-UC). Inflammatory bowel diseases such as CD, UC, and IC are distinguished from all other disorders, syndromes, and abnormalities of the gastroenterological tract, including irritable bowel syndrome (IBS).

“Risk variant” as used herein refers to genetic variants, the presence of which correlates with an increase or decrease in susceptibility to UC. Risk variants of UC include, but are not limited to variants at the PRDM1 genetic locus, such as “haplotypes” and/or a set of single nucleotide polymorphisms (SNPs) on a gene or chromatid that are statistically associated. More preferably, risk variants can include, but are not limited to rs2185379, rs9480625, rs573869, rs548234, and rs693612, or SEQ. ID. NO.: 1, SEQ. ID. NO.: 2, SEQ. ID. NO.: 3, SEQ. ID. NO.: 4, and SEQ. ID. NO.: 5, respectively. “MR-UC genetic risk variant” refers to genetic variants, or SNPs, that have an association with the MR-UC, or ulcerative colitis requiring colectomy, phenotype.

“Treatment” or “treating,” as used herein refer to both therapeutic treatment and prophylactic or preventative measures, wherein the object is to prevent, slow down and/or lessen the disease even if the treatment is ultimately unsuccessful. Those in need of treatment include those already with UC as well as those prone to have UC or those in whom UC is to be prevented. For example, in UC treatment, a therapeutic agent may directly decrease the pathology of UC, or render the cells of the gastroenterological tract more susceptible to treatment by other therapeutic agents.

As used herein, “diagnose” or “diagnosis” refers to determining the nature or the identity of a condition or disease. A diagnosis may be accompanied by a determination as to the severity of the disease. Diagnosis as it relates to the present invention, relates to the diagnosis of UC.

As used herein, “prognostic” or “prognosis” refers to predicting the probable course and outcome of UC or the likelihood of recovery from UC. The prognosis can include the presence, the outcome, or the aggressiveness of the disease.

As used herein, the term “biological sample” or “sample” means any biological material obtained from an individual from which nucleic acid molecules can be prepared. Examples of a biological sample include, but are not limited to whole blood, plasma, serum, saliva, cheek swab, urine, stool, or other bodily fluid or tissue that contains nucleic acid.

“Homology” refers to the percentage number of nucleic or amino acids that are identical or constitute conservative substitutions. Homology may be determined using sequence comparison programs such as GAP (Deveraux et al., 1984, Nucleic Acids Research 12, 387-395) which is incorporated herein by reference. In this way sequences of a similar or substantially different length to those cited herein could be compared by insertion of gaps into the alignment, such gaps being determined, for example, by the comparison algorithm used by GAP.

By “gene” is meant a unit of inheritance that occupies a specific locus on a chromosome and consists of transcriptional and/or translational regulatory sequences and/or a coding region and/or non-translated sequences (i.e., introns, 5′ and 3′ =translated sequences).

As used herein, the term “locus,” or “genetic locus” generally refers to a genetically defined region of a chromosome carrying a gene or any other characterized sequence.

The terms “patient” and “subject” are used interchangeably and refer to patients and subjects of human or other mammal and includes any individual it is desired to examine or treat using the methods of the invention. However, it will be understood that “patient” does not imply that symptoms are present. Suitable mammals that fall within the scope of the invention include, but are not restricted to, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes).

The term “polymorphism,” as used herein, refers to a difference in the nucleotide or amino acid sequence of a given region as compared to a nucleotide or amino acid sequence in a homologous-region of another individual, in particular, a difference in the nucleotide of amino acid sequence of a given region which differs between individuals of the same species. A polymorphism is generally defined in relation to a reference sequence. Polymorphisms include single nucleotide differences, differences in sequence of more than one nucleotide, and single or multiple nucleotide insertions, inversions and deletions; as well as single amino acid differences, differences in sequence of more than one amino acid, and single or multiple amino acid insertions, inversions, and deletions. A “polymorphic site” is the locus at which the variation occurs. It shall be understood that where a polymorphism is present in a nucleic acid sequence, and reference is made to the presence of a particular base or bases at a polymorphic site, the present invention encompasses the complementary base or bases on the complementary strand at that site.

The term “polynucleotide” or “nucleic acid” as used herein designates mRNA, RNA, cRNA, cDNA or DNA. The term typically refers to oligonucleotides greater than 30 nucleotide residues in length.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

By “single nucleotide polymorphism (SNP)” as used herein refers to a change in which a single base in the DNA differs (such as via substitutions, addition or deletion) from the usual base at that position. For example, a single nucleotide polymorphism is characterized by the presence in a population of one or two, three or four nucleotides (i.e., adenosine, cytosine, guanosine or thymidine) at a particular locus in a genome such as the human genome. It will be recognized that while methods of the present invention are directed to the identification of certain SNPs within the PRDM1 gene, the methods can be used to identify other PRDM1-associated SNPs either alone or in combination with the exemplified SNPs, or combined with methods for determining other PRDM1-associated polymorphisms to increase the accuracy of the determination.

As used herein, rs2185379, rs9480625, rs573869, rs548234, and rs693612 are accession numbers used to represent SNPs, or genetic variants. Examples of rs2185379, rs9480625, rs573869, rs548234, and rs693612 are described herein as SEQ. ID. NO.: 1, SEQ. ID. NO.: 2, SEQ. ID. NO.: 3, SEQ. ID. NO.: 4, and SEQ. ID. NO.: 5, respectively.

As used herein, “PRDM1” is an abbreviation for PR domain containing 1, with ZNF domain, a gene encoding a protein (BLIMP1) that acts as a repressor of beta-interferon gene expression, and acts to promote B cell maturation into plasma cells. Similarly, “BLIMP1” is an abbreviation for B-lymphocyte-induced maturation protein 1, a protein encoded by PRDM1.

As disclosed herein, the Blimp-1 expression profile was evaluated and tested as to whether regulation of T cell responses by Blimp-1 could influence intestinal mucosa homeostasis. Genotyping in well-characterized populations was performed using standard techniques. Standard statistical analyses tested for association. Expression of Blimp-1 mRNA measured by real time PCR in naïve (CD45RA+/HLADR−/CD25−) and memory/effector (CD45RA-) CD4+ and CD8+ T cells sorted from normal donors PBMC. For analysis of Blimp-1 expression in mucosal T cells, CD3+ T cells were purified by positive selection from the lamina propria (LP) from surgical intestinal specimens obtained from control individuals, CD or ulcerative colitis (UC) patients. All samples were obtained in accordance with the IRB approved protocol.

As further disclosed herein, an association was found between the PRDM1 locus and CD in the cohort of 1,683 CD cases and 1,049 non-IBD controls (p=0.014). Association was also demonstrated between colectomy in MR-UC and the PRDM1 locus (p=0.0029) in a case control study of 324 MR-UC patients with 537 non-MRF-UC. Blimp-1 expression in peripheral human T cells was found to be similar to that observed in mice, with naive (both CD4 and CD8) T cells expressing lower levels compared with effector/memory cells. Blimp-1 expression by CD3⁺ T lymphocytes from the intestinal LP in both control and IBD patients was also demonstrated. However IBD patients (both UC and CD) tend to show less expression compared to controls. The data is not in conflict with genetic associations previously seen and support a PRDM1 association with disease severity in UC. The expression studies further implicate Blimp-1 in IBD. In addition, an association was found between the SNP rs2185379 and IBD (Table 1).

Likewise, 4 out of 30 SNPs tested at the PRDM1 locus statistically showed significant association with ulcerative colitis, as compared to non-IBD controls (Table 2).

In one embodiment, the present invention provides a method of prognosing IBD in an individual by determining the presence or absence of a risk variant at the PRDM1 genetic locus, where the presence of a risk variant at the PRDM1 genetic locus is indicative of a severe form of IBD. In another embodiment, the presence of the risk variant at the PRDM1 genetic locus is determined by the expression of Blimp-1 protein and/or Blimp-1 mRNA. In another embodiment, the IBD is ulcerative colitis. In another embodiment, the severe form of IBD is MR-UC.

In one embodiment, the present invention provides a method of diagnosing an IBD subtype in an individual by determining the presence or absence of a risk variant at the PRDM1 genetic locus, where the presence of a risk variant at the PRDM1 genetic locus is indicative of the IBD subtype. In another embodiment, the presence of the risk variant at the PRDM1 genetic locus is determined by the expression of Blimp-1 protein and/or Blimp-1 mRNA. In another embodiment, the IBD subtype is ulcerative colitis. In another embodiment, the IBD subtype is MR-UC.

In one embodiment, the present invention provides a method of treating IBD in an individual by determining the presence of one or more risk variants at the PRDM1 genetic locus and treating the individual. In another embodiment, the IBD is MR-UC.

A variety of methods can be used to determine the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of a variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature, “Nucleic Acids Research 28:655-661 (2000)). Minor grove binders include, but are not limited to, compounds such as dihydrocyclopyrroloindole tripeptide (DPI,).

Sequence analysis also may also be useful for determining the presence or absence of a variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al.,(Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against CD in an individual may be practiced using one or any combination of the well known assays described above or another art-recognized genetic assay.

There are also many techniques readily available in the field for detecting the presence or absence of polypeptides or other biomarkers, including protein microarrays. For example, some of the detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Similarly, there are any numbers of techniques that may be employed to isolate and/or fractionate biomarkers. For example, a biomarker may be captured using biospecific capture reagents, such as antibodies, aptamers or antibodies that recognize the biomarker and modified forms of it. This method could also result in the capture of protein interactors that are bound to the proteins or that are otherwise recognized by antibodies and that, themselves, can be biomarkers. The biospecific capture reagents may also be bound to a solid phase. Then, the captured proteins can be detected by SELDI mass spectrometry or by eluting the proteins from the capture reagent and detecting the eluted proteins by traditional MALDI or by SELDI. One example of SELDI is called “affinity capture mass spectrometry,” or “Surface-Enhanced Affinity Capture” or “SEAC,” which involves the use of probes that have a material on the probe surface that captures analytes through a non-covalent affinity interaction (adsorption) between the material and the analyte. Some examples of mass spectrometers are time-of-flight, magnetic sector, quadrupole filter, ion trap, ion cyclotron resonance, electrostatic sector analyzer and hybrids of these.

Alternatively, for example, the presence of biomarkers such as polypeptides maybe detected using traditional immunoassay techniques. Immunoassay requires biospecific capture reagents, such as antibodies, to capture the analytes. The assay may also be designed to specifically distinguish protein and modified forms of protein, which can be done by employing a sandwich assay in which one antibody captures more than one form and second, distinctly labeled antibodies, specifically bind, and provide distinct detection of, the various forms. Antibodies can be produced by immunizing animals with the biomolecules. Traditional immunoassays may also include sandwich immunoassays including ELISA or fluorescence-based immunoassays, as well as other enzyme immunoassays.

Prior to detection, biomarkers may also be fractionated to isolate them from other components in a solution or of blood that may interfere with detection. Fractionation may include platelet isolation from other blood components, sub-cellular fractionation of platelet components and/or fractionation of the desired biomarkers from other biomolecules found in platelets using techniques such as chromatography, affinity purification, 1D and 2D mapping, and other methodologies for purification known to those of skill in the art. In one embodiment, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

Blimp-1, a transcriptional repressor encoded by the PRDM1 gene, is as a crucial regulator of T cell function. In mice, T-cell specific deletion of Blimp-1 (Blimp-1CKO) results in altered peripheral T cell homeostasis and development of a fatal inflammatory response targeting specifically the colon. Furthermore the Crohn's Disease (CD) genome wide association study (GWAS) meta-analysis identified association between a SNP (rs7746082) at the PRDM1 locus and CD susceptibility (p=2.44×10⁻¹⁰). However additional studies, such as functional and expression studies, are needed to implicate PRDM1 as the causative gene at this locus.

The Blimp-1 expression profile was evaluated and tested as to whether regulation of T cell responses by Blimp-1 could influence intestinal mucosa homeostasis. Genotyping in well-characterized populations was performed using standard techniques. Standard statistical analyses tested for association. Expression of Blimp-1 mRNA measured by real time PCR in naive (CD45RA+/HLADR−/CD25−) and memory/effector (CD45RA−) CD4+ and CD8+ T cells sorted from normal donors PBMC. For analysis of Blimp-1 expression in mucosal T cells, CD3+ T cells were purified by positive selection from the lamina propria (LP) from surgical intestinal specimens obtained from control individuals, CD or ulcerative colitis (UC) patients. All samples were obtained in accordance with the IRB approved protocol.

An association was found between the PRDM1 locus and CD in the cohort of 1,683 CD cases and 1,049 non-IBD controls (p=0.014). Association were also demonstrated between colectomy in MR-UC and the PRDM1 locus (p =0.0029) in a case control study of 324 MR-UC patients with 537 non-MRF-UC. Blimp-1 expression in peripheral human T cells was found to be similar to that observed in mice, with naïve (both CD4 and CD8) T cells expressing lower levels compared with effector/memory cells. Blimp-1 expression by CD3⁺ T lymphocytes from the intestinal LP in both control and IBD patients was also demonstrated. However IBD patients (both UC and CD) tend to show less expression compared to controls. The data is not in conflict with genetic associations previously seen and support a PRDM1 association with disease severity in UC. The expression studies further implicate Blimp-1 in IBD.

TABLE 1
Association of SNP rs2185379 with IBD Rs2185379 S74G

| | 0 | 1 | 2 | P fisher |
|---|---|---|---|---|
| CD | 595 | 34 | 0 | 0.01 |
| UC | 585 | 38 | 1 | 0.01 |
| Control | 569 | 17 | 1 | |
| IBD | | | | 0.009 |

UC glm β = 0.67, p = 0.015

TABLE 2
4 SNPs at PRDM1 locus associated with ulcerative colitis

| SNP | Case | Control | P |
|---|---|---|---|
| rs9480625 | 0.239 | 0.206 | 0.0039 |
| rs573869 | 0.773 | 0.739 | 0.0052 |
| rs548234 | 0.714 | 0.674 | 0.0017 |
| rs693612 | 0.402 | 0.363 | 0.0037 |

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive.

1. A method of prognosing ulcerative colitis (UC) in a subject, comprising:
obtaining a sample from the subject;
assaying the sample to determine the presence or absence of one or more risk variants at the PRDM1 genetic locus; and
prognosing a severe form of ulcerative colitis relative to a healthy individual based on the presence of one or more risk variants at the PRDM1 genetic locus.

2. The method of claim 1, wherein the severe form of UC is medically refractive ulcerative colitis (MR-UC).

3. The method according to claim 1, wherein the one or more risk variants are selected from the group consisting of SEC. ID. NO.: 1, SEC. ID. NO.: 2, SEQ. ID. NO.: 3, SEQ. ID. NO.: 4, and SEQ. ID. NO.: 5.

4. The method according to claim 1, wherein the presence of one or more risk variants at the PRDM1 genetic locus is determined by assaying for the expression of the Blimp-1protein and/or Blimp1 mRNA.

5. The method according to claim 1, wherein the sample is whole blood, plasma, serum, saliva, cheek swab, urine, or stool.

6. A method of diagnosing susceptibility to ulcerative colitis (UC) in a subject, comprising:
obtaining a sample from the subject;
assaying the sample to determine the presence or absence of one or more risk variants at the PRDM1 genetic locus; and
diagnosing a severe form of ulcerative colitis based on the presence of one or more risk variants at the PRDM1 genetic locus.

7. The method of claim 6, wherein the severe form of UC is MR-UC.

8. The method according to claim 6, wherein the one or more risk variants are selected from the group consisting of SEC. ID. NO.: 1, SEC. ID. NO.: 2, SEQ. ID. NO.: 3, SEQ. ID. NO.: 4, and SEQ. ID. NO.: 5.

9. The method according to claim 6, wherein the presence of one or more risk variants at the PRDM1 genetic locus is determined by assaying for the expression of the Blimp-1protein and/or Blimp1 mRNA.

10. The method according to claim 6, wherein the sample is whole blood, plasma, serum, saliva, cheek swab, urine, or stool.

11. A method of diagnosing susceptibility to medically refractive ulcerative colitis (MR-UC) in a subject, comprising:
obtaining a sample from the subject;
assaying the sample to determine the presence or absence of one or more MR-UC genetic risk variants; and
diagnosing susceptibility to MR-UC in the subject based on the presence of one or more MR-UC genetic risk variants.

12. The method according to claim 11, wherein the one or more risk variants are selected from the group consisting of SEC. ID. NO.: 1, SEC. ID. NO.: 2, SEQ. ID. NO.: 3, SEQ. ID. NO.:
4, and SEQ. ID. NO.: 5.

13. The method according to claim 11, wherein the presence of one or more risk variants at the PRDM1 genetic locus is determined by assaying for the expression of the Blimp-1protein and/or Blimp1 mRNA.

14. The method according to claim 11, wherein the sample is whole blood, plasma, serum, saliva, cheek swab, urine, or stool.

15. A method of treating a subject for UC, comprising:
determining the presence of one or more risk variants at the PRDM1 genetic locus; and
treating the subject;
wherein the one or more risk variants are selected from the group consisting of SEC. ID. NO.: 1, SEC. ID. NO.: 2, SEQ. ID. NO.: 3, SEQ. ID. NO.: 4, and SEQ. ID. NO.: 5.

16. The method of claim 15, wherein the presence of one or more risk variants at the PRDM1 genetic locus is determined by the expression of the Blimp-1 protein and/or Blimp1 mRNA.

17. The method according to claim 15, wherein the sample is whole blood, plasma, serum, saliva, cheek swab, urine, or stool.
